(19) United States
(12) Patent Application Publication
(10) Pub. No.: US 2008/0172778 A1
(43) Pub. Date: Jul. 24, 2008

(54) HELMET GOGGLE

(76) Inventor: Charles Lysogorski, Shelburne, VT (US)

Correspondence Address: SCHMEISER, OLSEN & WATTS, 22 CENTURY HILL DRIVE, SUITE 302, LATHAM, NY, 12110, US

(21) Appl. No.: 12/016,356

(22) Filed: Jan. 18, 2008

Related U.S. Application Data
(60) Provisional application No. 60/881,547, filed on Jan. 22, 2007.

(51) Int. Cl. A61F 9/02

(52) U.S. Cl. 2/436

(57) ABSTRACT

A goggle useful in sports and activities that is configured and dimensioned to have the top of the lens be spaced away from the forehead to prevent fogging within the goggle by trapping heat. The goggle may also be configured to rest upon the outside front portion of either a pre-existing or aesthetically matching helmet when spaced away from the forehead. A plurality of vents in either the seals or the lens may also further prevent fogging when used in conjunction with preventing the top seal from resting upon the forehead.

### CROSS REFERENCE TO RELATED APPLICATIONS

A claim of benefit is made to U.S. Provisional Application No. 60/881,547 filed Jan. 22, 2007, the contents of which are incorporated by reference in their entirety.

### BACKGROUND

Protective goggles have been used in many sports and activities. Originally many goggles were made of glass and were mainly for industrial protection such as for chemists or divers. As advances of materials and knowledge of safety by the public increased goggles were introduced into many sports and leisure activities. The goggles currently are mostly made of light weight plastics allowing for inexpensive use and comfortable use by both children and adults in most leisure activities.

### SUMMARY OF THE INVENTION

A goggle comprising: a lens configured to be spaced away from the forehead when worn to prevent condensation from sweat; a lower seal attached to said lens configured to space the lens away from the face and seal above the exposed nose and beneath the eyes; and an attachment device operatively attached to said lens so that pressure is maintained upon the lower seal to maintain the lens a minimum distance from the forehead.

A goggle configured for use with a pre-existing helmet comprising: an upper seal of the goggle dimensioned and configured to be operatively attached to an outside front portion of said pre-existing helmet; a lower seal of the goggle dimensioned and configured to seal above the exposed nose and beneath the eyes; a lens attached to said upper and lower seals, wherein said lens is spaced away from the forehead sufficiently to prevent condensation from forehead sweat; and an attachment device attached to the lens so that pressure is maintained upon the seals.

A rollable goggle configured for use with a helmet comprising: a helmet; an upper seal of the goggle dimensioned and configured to operatively attach to an outside front portion of the helmet to allow unhindered evaporation of forehead sweat; a lower seal of the goggle dimensioned and configured to seal above the exposed nose and beneath the eyes; a lens attached to said upper and lower seals, wherein said lens is spaced away from the forehead sufficiently to prevent condensation; a support element that engages said seal creating a plurality of spaces between said seal and said lens; an open cell foam within said plurality of spaces to vent said lens; an attachment device attached to the lens so that pressure is maintained upon the seals.

The foregoing and other features and advantages of the invention will be apparent from the following more particular description of embodiments of the invention. It is to be understood that both the foregoing general description and the following detailed description are exemplary, but are not restrictive, of the invention.

### BRIEF DESCRIPTION OF THE DRAWINGS

Some of the embodiments of this invention will be described in detail, with reference to the following figures, wherein like designations denote like members, wherein:

FIG. 1 displays an embodiment of goggle worn without a helmet with the adjustable strap in the lower position;

FIG. 2 displays an overhead perspective view of the goggle worn without a helmet with a spacer and with the adjustable strap in the upper position;

FIG. 3 displays an overhead rear perspective view of one embodiment of the goggle;

FIG. 4 displays an overhead side perspective view of one embodiment of a rollable goggle;

FIG. 5 displays a cross-sectional view of a dual lens;

FIG. 6 displays a cross sectional view of one embodiment of a rollable goggle seal;

FIG. 7 displays a front view of a lens having a plurality of vent holes;

FIG. 8 displays a goggle worn on a pre-existing moto-cross helmet;

FIG. 9 displays an embodiment of a goggle for use with a helmet having a frame;

FIG. 10 displays a goggle having a frameless ballistic grade protective lens;

FIG. 11 displays a rolled goggle and a rollable goggle in a storage bag;

FIG. 12 displays a side view of a goggle worn on a pre-existing ski helmet having a vent in the upper seal;

FIG. 13 displays a side view of a goggle worn on a pre-existing ski helmet without a vent in the upper seal;

FIG. 14 displays a overhead perspective view of a helmet with a removable vent for attaching the goggle thereupon;

FIG. 15 displays a structural element of a rollable goggle in an unrolled position;

FIG. 16 displays a structural element of a rollable goggle in a rolled position;

FIG. 17 displays a rollable foam block seal attached directly to the lens;

FIG. 18 displays a side view of a goggle/helmet combination with the goggles hingedly attached to a helmet;

FIG. 19 displays a side view of a goggle/helmet combination with the goggles hingedly attached to a helmet with the goggles in the raised position; and

FIG. 20 displays a front view of a goggle/helmet combination with the goggles hingedly attached to a helmet.

### DETAILED DESCRIPTION OF THE INVENTION

Although certain preferred embodiments of the present invention will be shown and described in detail, it should be understood that various changes and modifications may be made without departing from the scope of the appended claims. The scope of the present invention will in no way be limited to the number of constituting components, the materials thereof, the shapes thereof, the relative arrangement thereof, etc., and are disclosed simply as an example of an embodiment. The features and advantages of the present invention are illustrated in detail in the accompanying drawings, wherein like reference numerals refer to like elements throughout the drawings.

As a preface to the detailed description, it should be noted that, as used in this specification and the appended claims, the singular forms “a”, “an” and “the” include plural referents, unless the context clearly dictates otherwise.

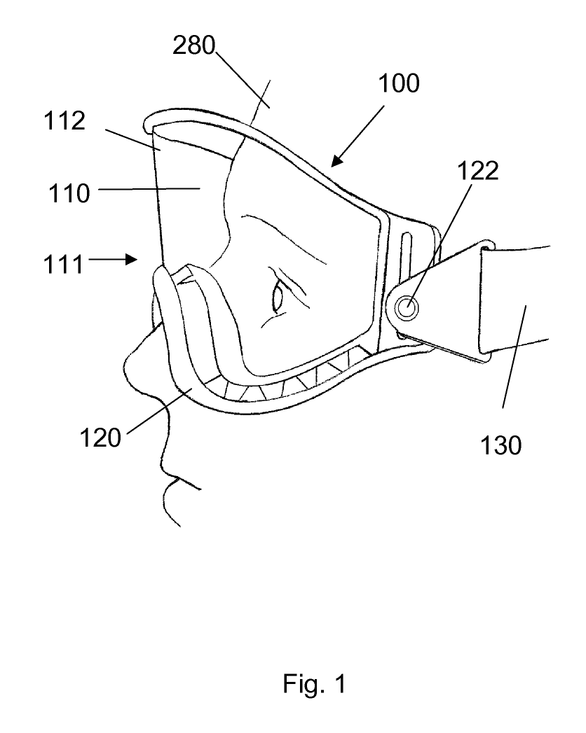

A goggle 100 as shown in FIG. 1 may comprise a lens 110 configured to be spaced away from the forehead 280 when worn helps to prevent condensation from sweat upon the lens 110. The spacing away from the forehead 280 of the lens 110 is made by the absence of a top seal that is configured to prevent resting against the forehead 280 so that it allows rapid evaporation while still providing superior protection from dust, wind and debris different from regular glasses.

A lower seal 120 is attached to the lens 110 and is configured so as to help space the top of the lens 112 away from the forehead 280 and seal 120 above the exposed nose and beneath the eyes so that it may prevent unwanted particles from entering the eyes from beneath by hindering and/or filtering unwanted or unfiltered airflow. An attachment device 130 is operatively attached either directly to the lens 110 or indirectly through a frame if present to the lens 110 so that bottom pressure 111 may be maintained upon the lower seal 120 to maintain a minimum distance from the forehead 280 of the top of the lens 112 to allow unhindered escape of moisture from the forehead 280.

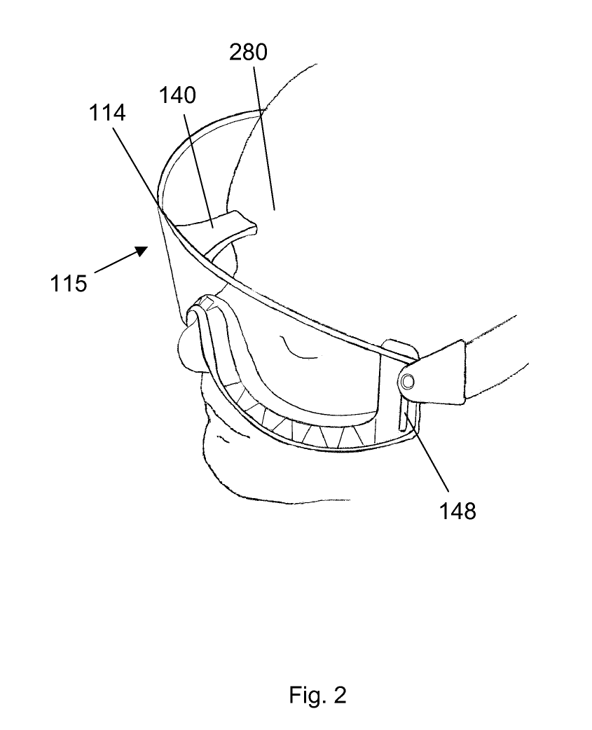

The goggle 100 may also further optionally comprise a center top spacer or a lens spacer 140 as shown in FIG. 2 that is operatively attached to an upper inside portion 114 of the lens 110 and resting against either the forehead 280 or the helmet 200 configured to allow evaporation of sweat and to space the lens 110 from the forehead 280 while allowing lower possible pressure against the bottom seal 120. The lens spacer 140 may be a small piece of foam, felt, portion of a frame or any other relatively incompressible material that may be a single piece positioned above the bridge of the nose or a plurality of pieces adjacent the temple or spaced allowing 95% or more of the inside portion 114 of the lens 110 to be free and allow unhindered space for evaporation. The lens spacer 140 may be an option for the wearer that desires to reduce pressure upon the lower seal 120 which may be controlled by the position of the attachment point 148 and could either be fixed, removable or retractable if undesired at a later point such as a kick stand that may be folded in either direction or to rest against the helmet 200 or a removable pad. In FIG. 1 the attachment point is positioned to maximize pressure on the lower seal 120 and FIG. 2 shows the positioning so that a majority of upper pressure 115 may be placed on the lens spacer 140.

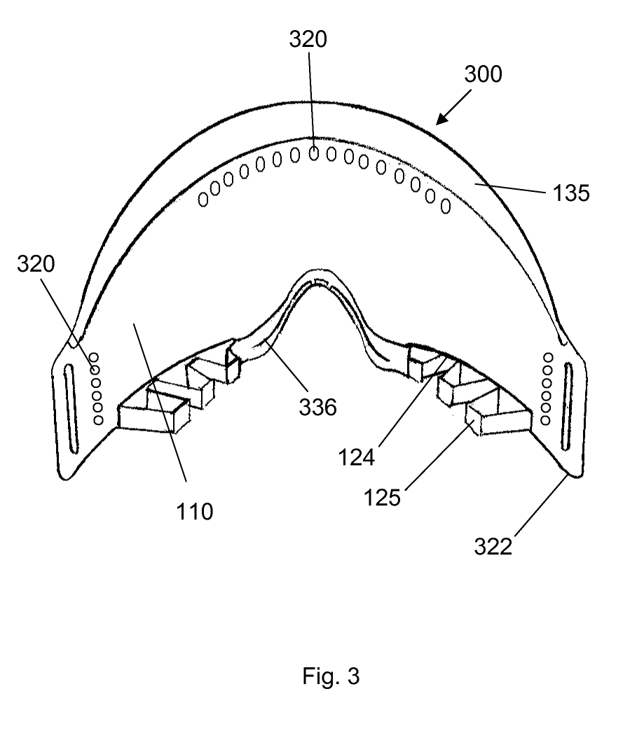

Another embodiment is goggle 300 as shown in FIG. 3 that may further comprise an upper seal 135 that is configured and dimensioned to rest and conform against an outside frontal portion of a pre-existing helmet 200 while being worn leaving the forehead 280 free to evaporate unhindered. The goggle 300 may also be configured to be used in conjunction with a specially made helmet 200 that is only modified to the extent necessary from a pre-existing helmet 200 that it is more aesthetically pleasing by having a recessed portion to match the goggle with the helmet when worn in conjunction with the goggle 300 and in addition by either color coordination or sculpting of exterior lines of the helmet and goggle to be in a more aesthetically pleasing shape, but still functionally similar to the use of the goggle 300 with a seal against the front outside portion of a pre-existing helmet 200.

The upper seal 135 of goggle 300 may also be configured to be removable so that it can revert to the top seal free goggle 100 if the pre-existing helmet 200 is removed if desired. The upper seal 135 when positioned against said outside frontal portion of a pre-existing helmet 200 still allows evaporation of sweat away from the goggles 300 and into the helmet 200 releasing heat from the forehead 280 without fogging the lens 110. The release of the forehead 280 heat into the helmet 200 may add to much greater comfort and satisfaction by the top seal 135 not acting as a sweatband and wicking moisture into the lens 110 as may occur with a conventional goggle having a forehead seal. The upper seal 135 shown in FIG. 3 may be foam, felt or other semi-compressible soft material that may be attached directly to the lens 110 and may compress firmly against the outer portion of the helmet 200 allowing for a lower profile, but with limited ventilation. The goggle 300 may also include a plurality of holes 320 in the upper portion of the lens 110 above the line of sight to allow additional airflow from behind the lens 110 while still maintaining a low profile with the helmet 200.

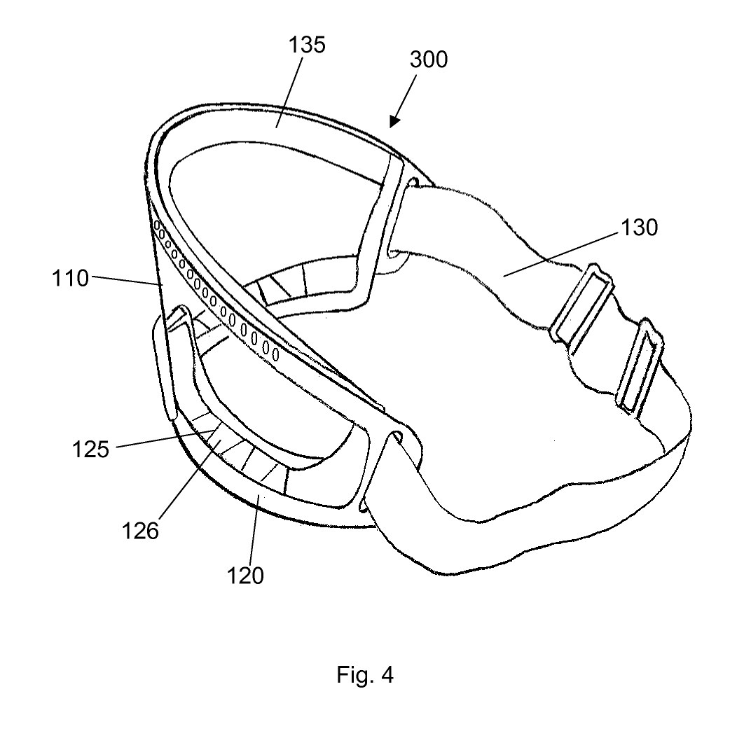

The goggle 300 may further comprise the element of a collapsible member 125 contained within seals 120, wherein said collapsible member 125 allows airflow. The collapsible member 125 may be plastic or other flexible material that can withstand the tension of a strap without substantially deflecting, but will deflect if a severe impact is made upon the goggle 300 to prevent injury. The collapsible member 125 is collapsible as described for purposes of both impact and rolling purposes because if struck frontally it prevents injury and if the goggle 300 is rolled the members 125 touch each other eliminating the space 124 positioned there between the members 124. The goggle 300 shown in FIG. 3 has a lens 110 that also acts as a frame where the upper seal 135 and the lower seal 120, which as shown in FIG. 4 may be formed by covering the collapsible member 125 with either an open or closed celled foam are configured to be used in conjunction with a pre-existing helmet 200 that may have been previously purchased by the sports enthusiast without the specific intention for use with the invention's goggle 300.

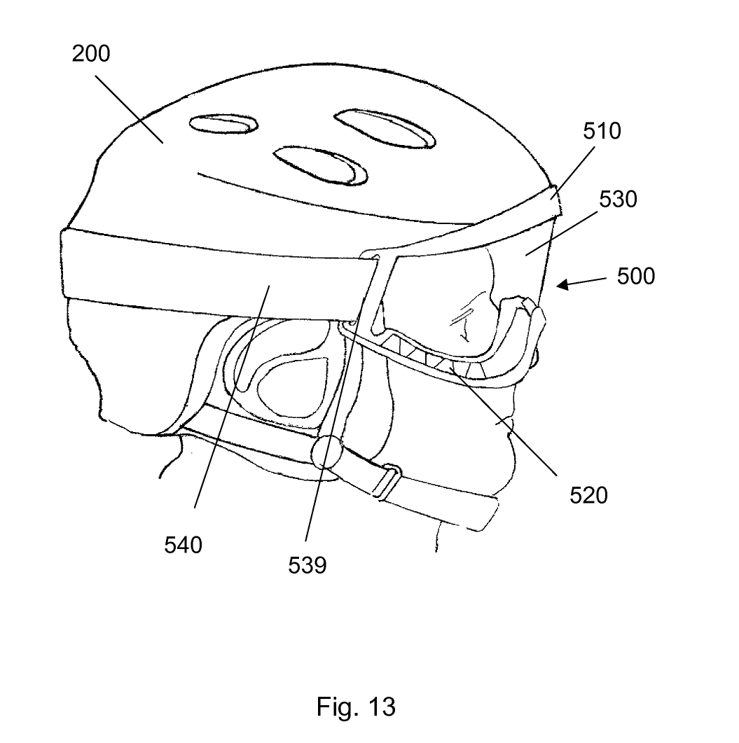

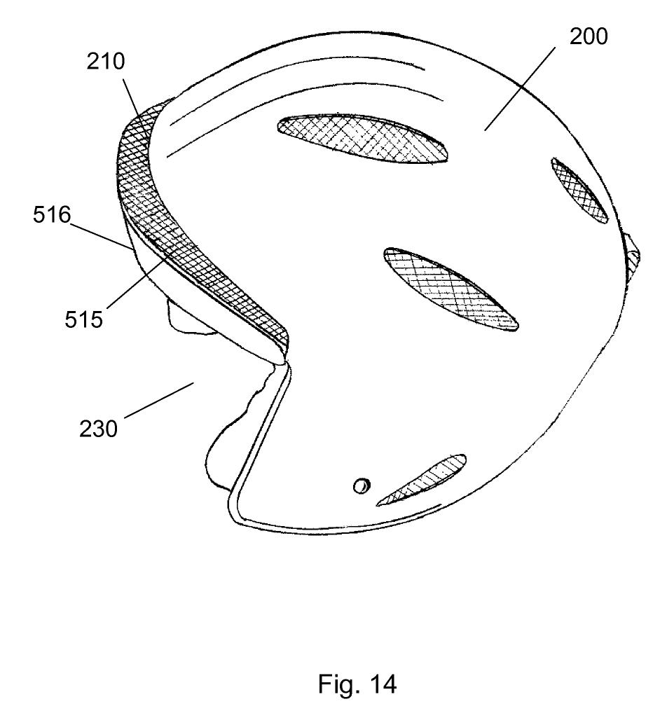

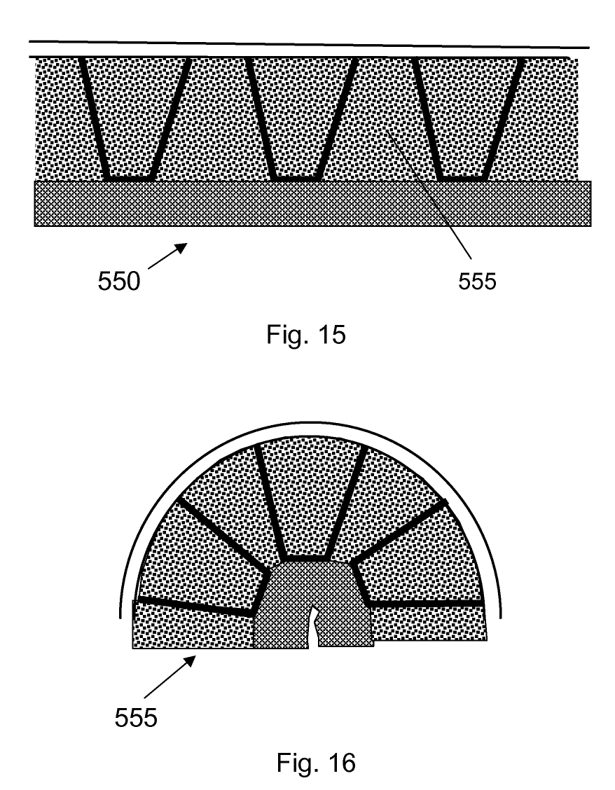

The collapsible member 125 allows the lens 110 to be properly spaced from face to match the offset from the outside edge of the helmet 200 as shown in FIG. 13. The collapsible member 125 may be a plurality of triangular or even round shaped profiles that are spaced and shaped to fold together allowing the frameless goggle 300 to be rolled as shown in FIGS. 15 and 16. The collapsible members 125 allow the goggle 300 to roll by flexing at the trough 124 positioned between the collapsible members 125 that allows bending of the goggle 300 until the sides 126 of the collapsible member 125 can touch.

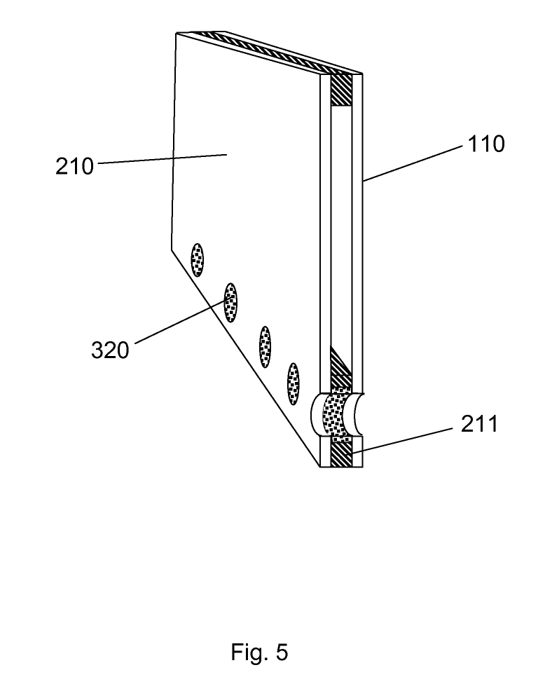

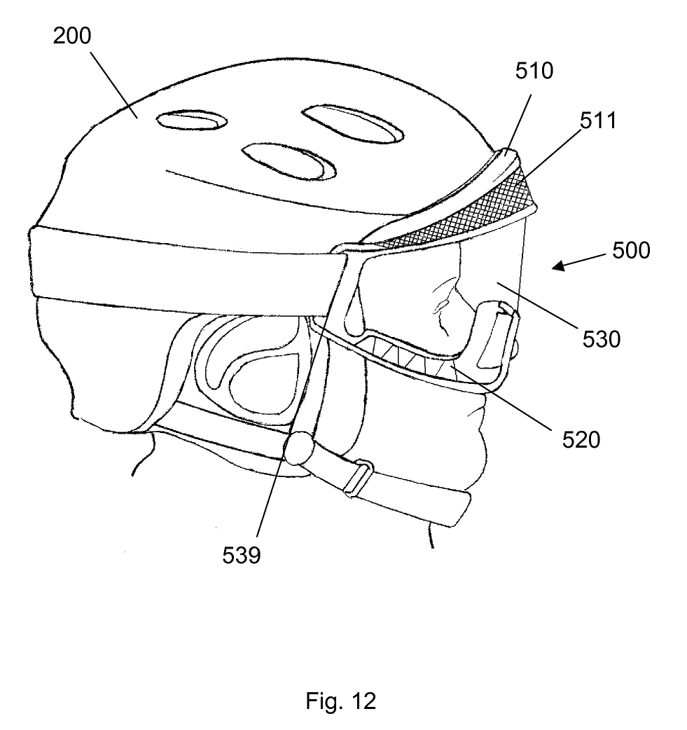

The goggle 500 as shown in FIG. 12 may be modified to further comprise vents 511 formed in said upper seal 510 positioned between the lens 530 and the front of the pre-existing helmet 200. The vents 511 may allow for further cooling and prevention of fogging in comparison to the non-vented top seal 510 as shown in FIG. 13. The non-vented top seal 510 as shown in FIG. 13 may be either a soft, but incompressible material such as a felt or a compressible material such as a closed cell foam either alone or felt coated. The goggle 500 may also further comprise in addition to the vent in the seal 510 or as shown in FIG. 5 a vent 320 in the lens 110 and a second parallel lens 210 positioned adjacent to the lens 110 to provide additional anti-fog protection. The dual pane lens 210 has a second pane 210 that is sealed 211 and parallel to the first or single lens 110 that further may prevent condensation by acting as an insulator.

The bottom seal 120 of the goggle 300 as shown in FIG. 4 may have an open celled foam 126 covering the collapsible member 125 when venting is desired or a closed cell foam if no venting is desired. The open celled foam 126 may allow for the filtration of dust and debris while still allowing movement of air. The bottom seal 120 may have an open celled foam 126 covering the collapsible member 125 and a closed cell foam may be positioned to be against the face of the wearer in combination with the open celled foam covering the openings that face downwards in the center of the members 125. There may be a soft top seal 135 configured to the front and the side of the helmet 200.

The top seal 135 on the goggle 300 may be attached directly upon the lens 110 by an adhesive. The top seal 135 rests against the front of the existing helmet 200 to ensure that the lens 110 is spaced away from the forehead 280. The top seal 135 is configured differently from the seal of a conventional goggle by being angled and positioned to rest upon a helmet 200 and not the forehead 280 as configured with a conventional goggle. To ensure that the goggle 300 has the top seal 135 positioned properly on the outside of the helmet 200 the lens 110 may be wider and longer than that of conventional goggle, which is dimensioned to fit within the opening 230 of the helmet 200 whereas the goggle 300 is dimensioned to seal and fit over the opening 230 of the helmet 200 and against the front portion of the helmet 200 instead of the forehead 280. Furthermore, the top seal 135 may be angled in some application to form a better seal against the outside front portion of the helmet 200 whereas the conventional goggle has a seal that is parallel to the forehead 280 and may not be angled. The goggle 300 has an additional benefit from the construction and placement of the lens 110 that allows for a broader field of view and prevention of fogging that may not available with a conventional goggle. Convention goggle rest against the forehead 280 within the opening 230 because they are smaller and closer to the face because it is required to be fit within the opening of the pre-existing helmet 200, whereas the instant goggle 300 rests outside and atop of the helmet opening 230 in helmets 200.

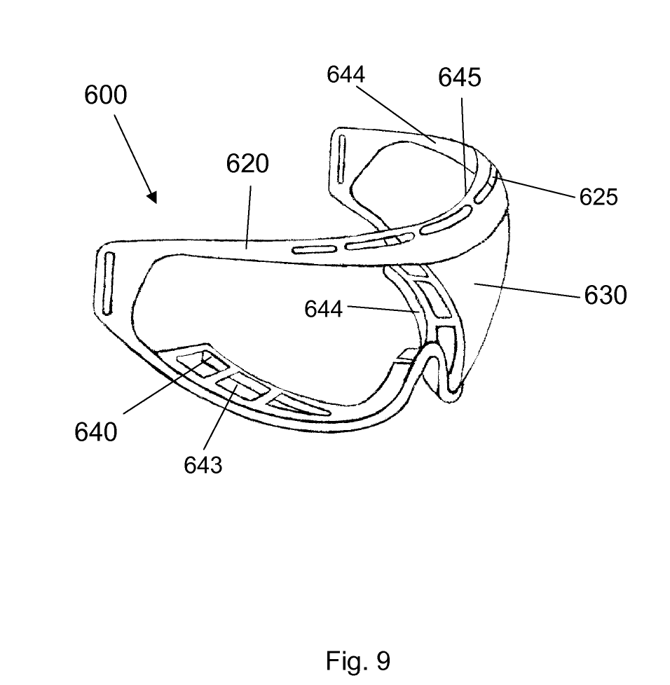

A conventional goggle upper frame and seal may fit to the face of the user around 0 degrees to the forehead 280. Helmet goggles of the instant may need an angled upper seal and/or frame, which is mated to the profile 210 of the helmet 200. Existing helmets 200 may typically have a 30 to 45 degree taper to the vertical (or forehead) on the helmet outer front surface 210. In FIG. 4 the thin flat seal 135 is used in conjunction with the vent frame 516 which may be attached to the helmet 200 (or custom helmet vent frame) which provides a matched sealing surface angle. In the embodiment of FIGS. 9,12 the upper seal 644, 510 and frame 620 may be angled to more effectively mate onto the angled portion 210 of the helmet 200. By angling the seal 135, 644, 510, and or frame 620 may provide more sealing of the helmet goggle 100, 300, 600 to the helmet 200 it also may create a cupping of the seal to the front of the helmet 200 thus securing the fit.

The lower seal of a conventional goggle may be made to fit the profile of the users nose cheeks and sides of the face around the eyes, which curves inward from the helmet 200. The helmet goggle 100, 300, 500, 600 follows the contour of the face like a conventional goggle until it reaches the helmet sides 210. The helmet 200 may then include a 20 mm to 40 mm profile away from the face. The helmet goggle 100, 300, 500, 600 may follow this jump in profile (FIGS. 3,8) to better allow the lens and or frame to be supported and seal on the sides 210 of the helmet 200. By keeping the seal and frame thin on the side of the helmet it may provide a more unrestricted horizontal field of vision. This also provides a proper radius of the lens 110 and frame 620 to allow maximum pressure of the seals to be applied to the face and helmet 200 by the securing device 130.

The goggle 600 as shown in FIG. 9 has a frame 620 that is attached to the lens 630. The seals 640, 645 are attached to the frame 620, which may then be covered by a soft material 644. The frame 620 houses the lens 630 as typical with a conventional goggle where the frame 620 and not the seal supports the tension of the instant goggle. The frame 620 of the goggle 600 is modified to allow sealing against the front and edge of a pre-existing helmet 200 instead of against the forehead 280 within the helmet opening 230 by providing a frame 620 that may be much wider and longer than a conventional frame. Furthermore, the frame 620 may be different with a much thinner upper seal 645 and thinner upper frame vent portion 625 of the goggle 600 in relation to the much thicker lower seal 640 to keep the lens 630 properly positioned or the upper frame vent portion 625 may be configured and positioned to be used in conjunction with an angled upper seal 645 that seals better against the outside portion of the helmet 200. The goggle frame 620 may be injection molded out of any relatively rigid and tough plastic as most goggle frames are made, which may contain holes or openings in either the top or bottom of the frame for venting as shown.

The goggle 600 may have an open celled foam 126 that covers the lower seal 640 allowing filtered airflow through the openings 643 molded into the lower seal portion 640 of the frame 620 or a closed cell foam as on seal 645 to prevent drafts. The lower seal 640 is dimensioned to be thick enough to keep the lens 630 properly positioned with respect to the face when the top seal 645 rests against the helmet 200. Attached either to the lower seal portion of the frame 640 or to the open celled foam 126 may be a soft felt like member 644 for comfort when resting against the face or sealing when resting against the helmet 200. A plurality of frame vents 625 may be placed on the frame 620 to minimize the profile of the goggles 600 when worn on the helmet 200.

The goggle 300 as shown in FIG. 3 may further comprise a vent 320 that consists of a plurality of holes formed in the lens 110 and also may be present in the lower seal 120 to allow airflow. The goggle 300 may further comprise a rigid portion 125 in said seals 120 to position the lens 110 a proper distance from the face and eyes. The goggle 300 shown in FIGS. 3 and 4 is frameless so that all structural load is support by the lens 110 and the seals 120 are attached directly to the lens by either adhesives or mechanical fasteners. The attachment device 130 is operatively attached directly to the lens 110 via either laced through a slot in the end 322 allowing the attachment device 130 such as an elastic strap to pass through the lens 110 or a mechanical device 122 as shown in FIG. 1 such as a screw, rivet, hinge or other type fastener that may or may not be slidably adjustable to tailor individual seal pressures. The substructure to support the seals directly onto the lens 110 are a rigid portion 125 in said seals 120 to position the lens 110 a proper distance from the face and a nose piece 336 to support the foam seal. The foam seal 120 may either be glued, mechanically fastened or injection molded directly over the rigid portion 125.

A goggle 500 as shown in FIGS. 12 and 13 is specifically configured for use with either a pre-existing helmet 200 or aesthetically matching helmet 200 by comprising an upper seal 510 of the goggle 500 dimensioned and configured to be operatively attached to an outside front portion 210 of said pre-existing helmet 200. The goggle 500 may be configured from either a framed or frameless type of setup where the upper seal 510 is shaped and dimensioned to seal against the outer front portion of the helmet 200, whereas a typical goggle has the seal resting upon the wearer's forehead 280 even if worn with a pre-existing helmet because it is dimensioned to be smaller than the facial opening 230 of a pre-existing helmet 200.

The goggle 500 of the instant invention has a frame and/or lens that is larger and/or wider than the facial opening 230 of a pre-existing helmet 200 so as to seal upon the outer edge and front portion 210 of the helmet 200. The top seal 510, of the dimensionally wider and taller lens/frame is configured to be positioned on the outer front portion 210 of a pre-existing helmet 200 thereby preventing trapping of forehead 280 sweat or heat. The top seal 510 by being positioned on the outer front portion of a pre-existing helmet 200 may either be much thinner or angled in comparison to a conventional top seal that rests upon on a forehead 280 and is configured to properly position the lens from the eyes in conjunction with the lower seal 520 of the goggle 500 that is dimensioned and configured to seal above the exposed nose and beneath the eyes. The difference in thicknesses between the upper 510 and lower seals 520, if any, is to allow for the offset from the forehead 280 of the user and position the top seal 510 on the outer front portion 210 of a pre-existing helmet 200 while the lower seal is still positioned against the upper cheeks below the eyes and over the nose. The goggle 500 may optionally have a top seal 510 that may be made of a thin felt or foam without a provision for a vent. The upper seal 510 is not on the forehead 280 allowing escape of heat and preventing trapping of perspiration from within the inner goggle space.

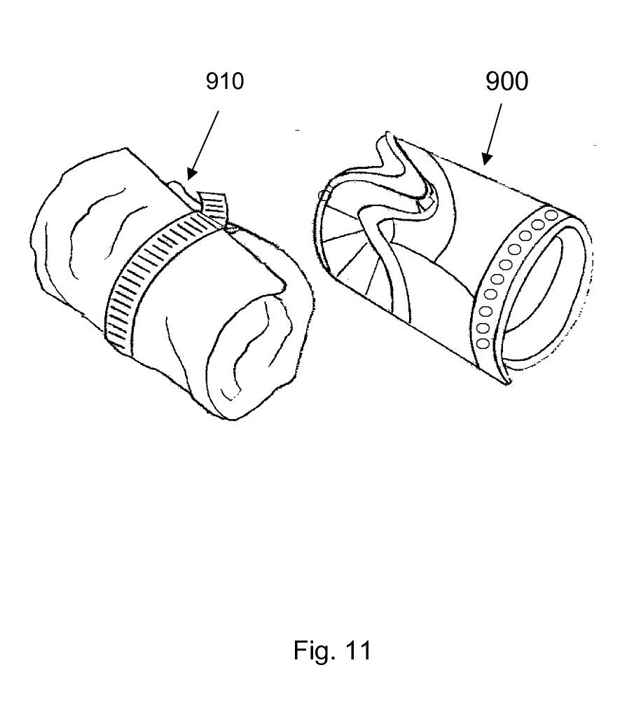

In a frameless embodiment of the goggle 500 a lens 530 is attached to said upper 510 and lower seals 520, whereas the embodiment of the goggle 500 with a frame would have the seals attached to the frame, which is then attached to the lens 530. The goggle 500 in a frameless configuration has a lens 530 that may use an adhesive to directly bond the seal to the lens 530. A goggle having a frame is too rigid to be rolled, but with a single planar lens 510 and special seal 550 the goggle may be rolled for storage 900 and placed into a goggle bag 910 in a rolled position as shown in FIG. 11.

The goggle 500 may have an attachment device 540 attached to the lens 530 so that pressure is maintained upon the seals 510, 520 as shown in FIGS. 12 and 13. The attachment device 540 is secured to the lens 530 by a tension providing device that may be an elastic strap having an adjustable length band that is looped through a slot 539 on either the lens or the frame that maintains pressure through elasticity properties of the band 540. The attachment device 540 attached to the lens 530 may be an elastic band that is mechanically coupled with a screw or pivot to either the lens 530 or the frame.

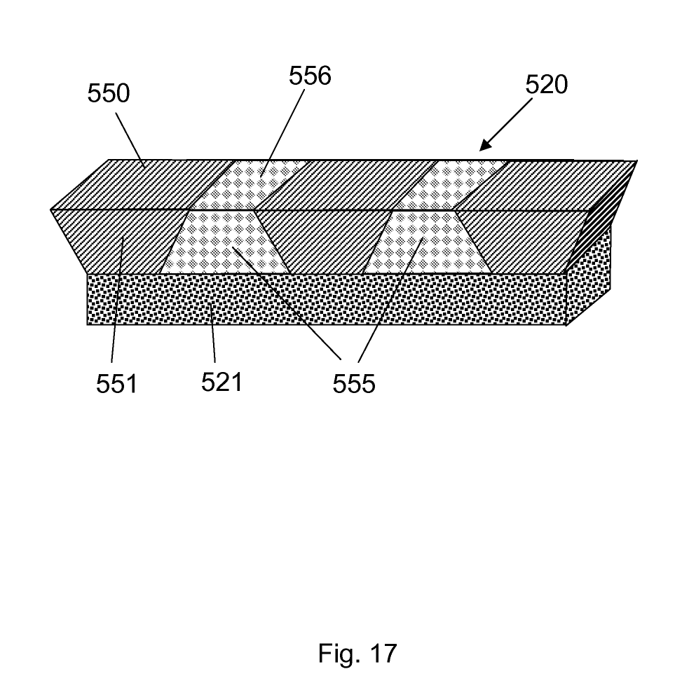

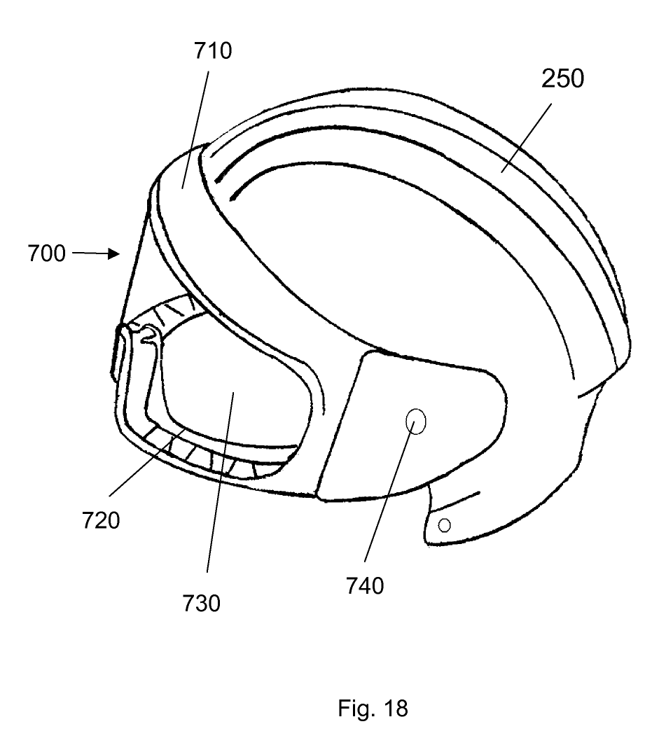

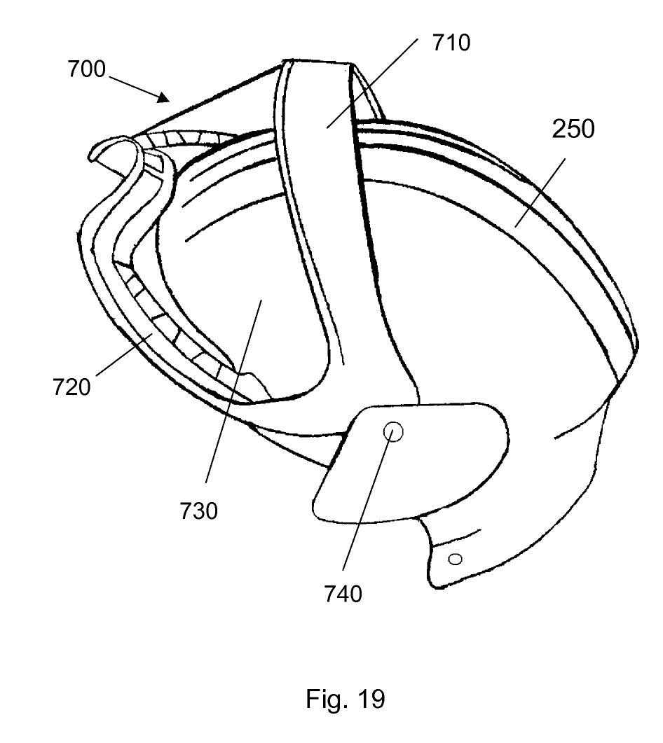

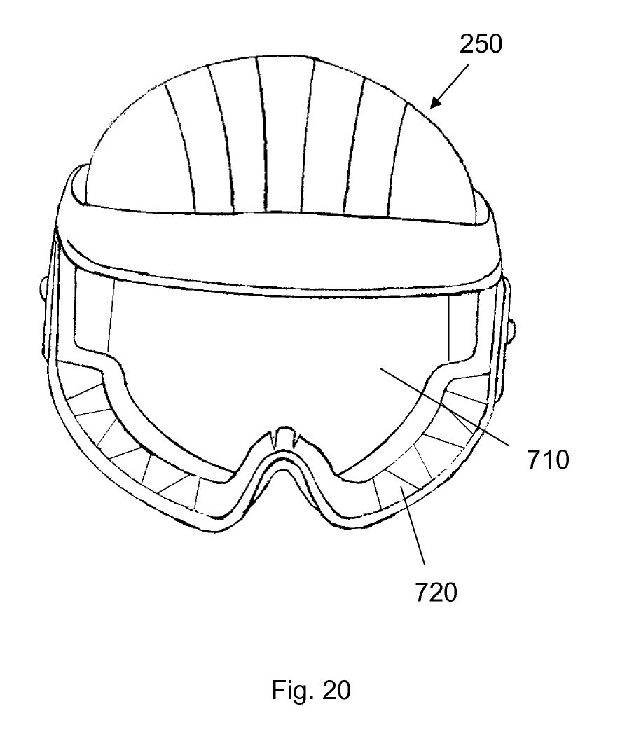

Another embodiment of the goggle is shown in FIG. 18 with a helmet goggle 700 that is modified so that the attachment device 740 hingedly holds the lens 730 onto a helmet 250 that is specifically configured for use with the goggles 700. The attachment device 740 may be a mechanical fastener that acts as a hinged pivot 740 so that pressure is maintained upon the seals 710, 720 when the lens 730 is in a closed position. The upper seal 710 is modified for use with a hinged lens 730 by being angled to slidingly seal against the front of a specially configured helmet 250. The upper seal 710 may be a compressible closed foam that would prevent the unwanted entry of water from rain or snow. The lower seal 720 when the lens 730 is in the lowered position is configured to rest above the nose and below the eyes as with the other goggles 300 of the invention. As shown in FIG. 20 the lower seal may optionally be ventilated with the seal 550 of FIG. 15 or the foam blocks of the seal 550 in FIG. 17 or alternatively just a solid piece of foam or felt as a seal without a vent.

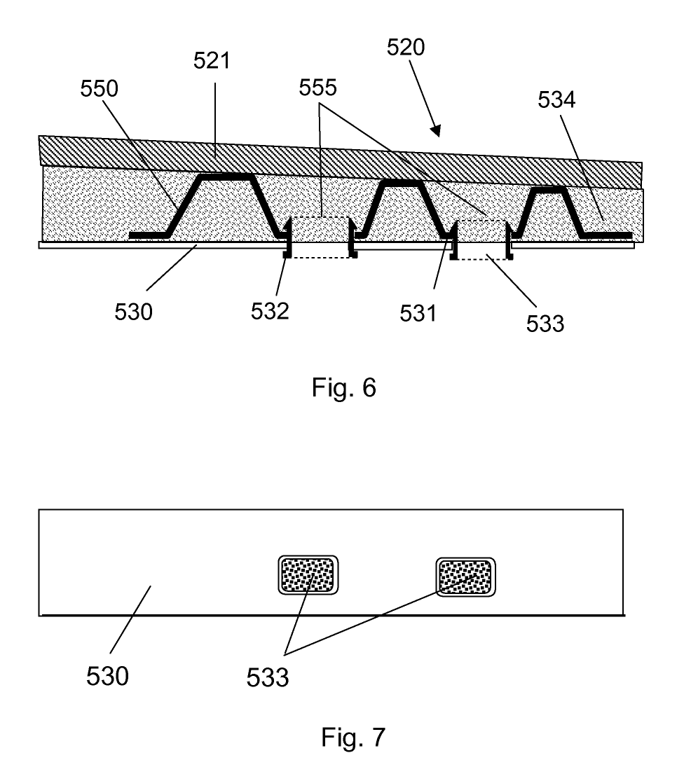

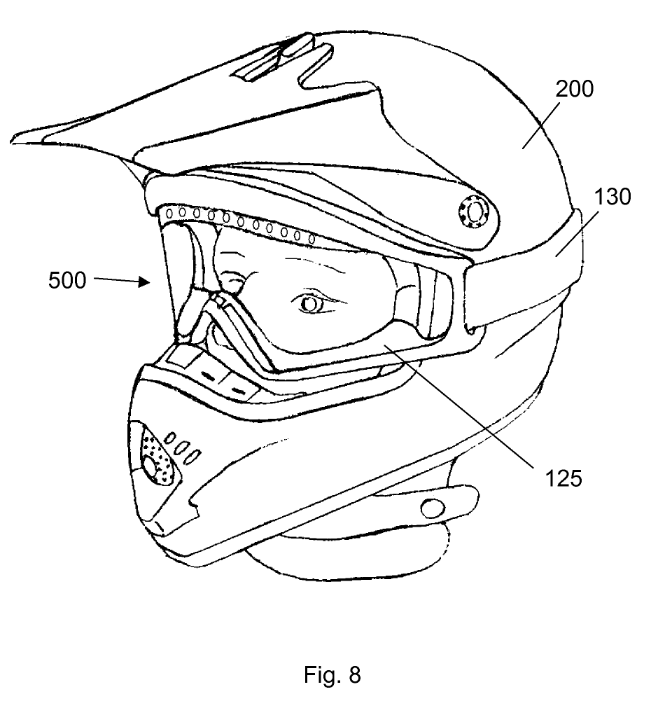

The goggle 500 as discussed above may also further comprise a vent 533 positioned on said lens 530 as shown in FIGS. 6 and 7 that may be an integral part of holding the seal material onto the lens 530. The goggle 500 when configured to be both frameless and rollable may further comprise an inwardly collapsible member 555 within said lens vented seals 533. The structure 550 in FIG. 6 has rigidity from either a plastic or flexible metal ribbon scaffold or band to support and position the lens 530 and an opening 534 filled with an open celled foam that may act as a vent and may be compressible to allow rolling of the goggles 500 for storage. The goggle 500 may have a second type of collapsible member 555 as shown in FIG. 17 that may comprise a first member 551, which is a rigid relatively stiff structural foam truncated triangular block within said seals comprising a plurality of structural supports 550 and a second member 555 within said seals adjacent said first member or a combination of both types of rigid elements 125, wherein said second member 555 comprises a plurality of open celled foam blocks which may act as a vent and a filter and compresses allowing rolling of the goggles for storage.

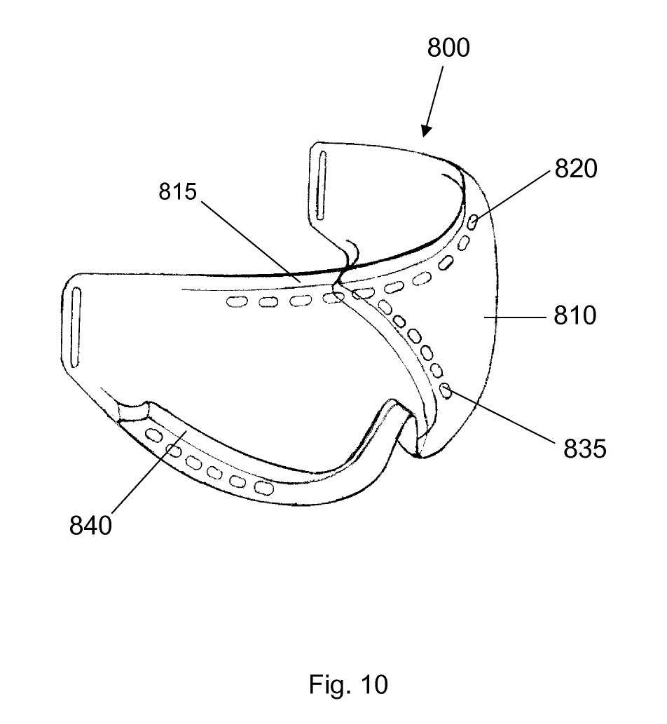

In another embodiment the goggle 800, as shown in FIG. 10 has a spherical lens 810 that is of a sufficient strength for use as a ballistic grade eye protection, which is shown in a frameless configuration, but may be used with a frame also. The ballistic grade lens 810 may be made of a polycarbonate or other light transmissible material that has properties to prevent injury from ballistic objects that may be from being either a desired thickness or material grade typically used in ballistic lenses 810. The ballistic goggle 800 may be configured to be worn either with or without a helmet 200 with the top portion of the lens 815 free of any seal or air flow obstruction from the forehead 280 as shown in FIG. 1 when used with the adjustable strap. Either embodiment of the ballistic goggle 800 would include a lower seal 840 that would properly space the goggle 800 away from the face and the forehead 280. The ballistic goggle 800 may also be configured to have a top seal 815 adhered onto the lens and dimensioned to seal against the front portion 210 of a helmet 200 to allow unhindered sweat evaporation from the forehead 280. The ballistic goggle 800 may also include upper lens vents 820 and lower lens vents 835 to allow for increased airflow and further prevent fogging.

The pre-existing helmet 200 may be used with a goggle 500 having an upper seal vent 511 positioned within the upper seal 510 to allow additional ventilation. A goggle 500 that has an upper seal 510 without a vent may be used in conjunction with a removable helmet vent 515 that is attached to the front portion 210 of a pre-existing helmet. The removable helmet vent 515 may be affixed either permanently or removably to the front portion 210 of a helmet 200 and is configured as shown in FIG. 15 or FIG. 17 to allow ventilation while not fully compressing when engaged by the goggle 300 without an upper seal upon the outer surface 516 of the removable vent 515.

The goggle 300 may further comprise a vent 320 formed in said lens 110 to allow airflow as shown in FIG. 3. The goggle 600 having a frame 620 may also further comprise either in combination or alone a vent consisting of a plurality of holes 643 formed in said lower seal 640. The plurality of holes 556 may be in the form of an open celled foam that also provides the function of filtration by preventing dirt and dust from passing large openings.

A rollable goggle 300, 500 of the embodiments shown in FIGS. 3-4 and 11-12 are configured for use with a pre-existing helmet 200. The goggle 300, 500 comprises an upper seal 135, 510 that is dimensioned and configured to operatively attach to an outside front portion 210 of a pre-existing helmet 200 to allow unhindered evaporation of forehead 280 sweat. The sealing on the outside of the helmet 200 prevents absorption of sweat by the top seal 135 and 510, which is one cause of fogging and additionally allows evaporation from the face to the top of the head without being confined between the seals of the lens 110, 530.

The rollable goggle 300, 500 has a lower seal 120, 520 as shown in FIGS. 4 and 12-13 that are dimensioned and configured to seal above the exposed nose and beneath the eyes. A lens 110, 530 is directly attached to the upper seal 135, 510 and lower seals 120, 520, wherein said lens 110 and the upper seal 135, 510 is spaced away from the forehead sufficiently to prevent condensation on the lens 110. The rollable goggle 300, 500 provides a storability, comfort, unobstructed field of vision and versatility not found in other goggles.

A support element 550 as shown in FIGS. 15-17 is used to provide structural support to the bottom seal 120 and occasional vented top seal 511 of the goggle. The support element 550 engages said seal 520 creating a plurality of spaces 555 between and within said seal 520 and said lens 110. An open cell foam 556 may be within said plurality of spaces 555 to vent said lens 110 and to prevent entry of dirt and debris.

The rollable goggle 300, 500 may further comprise at least one opening 533 in said lens 530 adjacent said seal 520 and a clip 532 for securing through at least one opening 533 in said lens 530 for securing said seal 520 to said lens 530. The seal 520 may contain the rigid plastic portions 550 that are encompassed by an open celled foam to allow for air flow and possible dirt filtration. An outer portion of the seal 521 is configured to be placed against the user's face and therefore should be comfortable by using for example a felt or soft foam, which may be adhered or attached to the structural portion 550. The plastic structural portions 550 have openings 531 within their bases 534 to mechanically engage a clip 533 through lens 530.

Various modifications and variations of the described apparatus and methods of the invention will be apparent to those skilled in the art without departing from the scope and spirit of the invention. Although the invention has been described in connection with specific embodiments, outlined above, it should be understood that the invention should not be unduly limited to such specific embodiments. Various changes may be made without departing from the spirit and scope of the invention as defined in the following claims.

What is claimed is:

1. A goggle comprising:
a lens configured to be spaced away from the forehead when worn to prevent condensation from sweat;
a lower seal attached to said lens configured to space away from the face and seal above the exposed nose and beneath the eyes; and
an attachment device operatively attached to said lens so that pressure is maintained upon the lower seal to maintain a minimum distance from the forehead of the lens.

2. The goggle of claim 1 further comprising:
a lens spacer operatively attached to an upper inside portion of the lens for spacing away the lens from the forehead being configured to allow evaporation of sweat.

3. The goggle of claim 1 further comprising:
an upper seal of the goggle configured to compress against an outside frontal portion of a pre-existing helmet while being worn, wherein the upper seal when positioned against said outside frontal portion of a pre-existing helmet allows evaporation of sweat from the forehead without fogging said lens.

4. The goggle of claim 3 further comprising:
a collapsible member contained within both said seals, wherein said collapsible member allows airflow.

5. The goggle of claim 1 further comprising:
a frame attached to said lens, wherein said seals and said elastic attachment device are attached to said frame.

6. The goggle of claim 3 further comprising:
a vent formed in said upper seal.

7. The goggle of claim 1 further comprising:
a second parallel lens positioned adjacent to the lens to provide additional anti fog protection.

8. The goggle of claim 1 further comprising:
a vent formed in said lens to allow airflow.

9. The goggle of claim 1 further comprising:
a vent consisting of a plurality of holes formed in said lower seal to allow airflow.

10. The goggle of claim 4 further comprising:
a rigid portion in said seals to position said lens.

11. A goggle configured for use with a helmet comprising:
an upper seal of the goggle dimensioned and configured to be operatively attached to an outside front portion of said helmet;
a lower seal of the goggle dimensioned and configured to seal above the exposed nose and beneath the eyes;
a lens attached to said upper and lower seals, wherein said lens is spaced away from the forehead sufficiently to prevent condensation from forehead sweat; and
an attachment device attached to the lens so that pressure is maintained upon the seals.

12. The goggle of claim 11 further comprising:
a recessed portion on said outer portion of said helmet that makes said lens flush with an exterior line of said helmet.

13. The goggle of claim 11 further comprising:
a collapsible member within said seals, which acts as a vent and allows rolling of the goggles for storage.

14. The goggle of claim 11 further comprising:
a first member within said seals comprising a plurality of structural supports; and
a second member within said seals adjacent said first member, wherein said second member comprises a plurality of open celled foam blocks which acts as a vent and a filter and compresses allowing rolling of the goggles for storage.

15. The goggle of claim 11 wherein the lens is of a ballistic grade eye protection.

16. The goggle of claim 11 further comprising:
a vent formed in said lens to allow airflow.

17. The goggle of claim 11 further comprising:
a vent consisting of a plurality of holes formed in said lower seal.

18. The goggle of claim 11 further comprising:
a slot on said goggle connected to said attachment device, said device positionable within said slot to adjust pressure on either seal.

19. A rollable goggle configured for use with a pre-existing helmet comprising:
an upper seal of the goggle dimensioned and configured to operatively attach to an outside front portion of a pre-existing helmet to allow unhindered evaporation of forehead sweat;
a lower seal of the goggle dimensioned and configured to seal above the exposed nose and beneath the eyes;
a lens attached to said upper and lower seals, wherein said lens is spaced away from the forehead sufficiently to prevent condensation;
a support element that engages said seal creating a plurality of spaces between said seal and said lens;
an open cell foam within said plurality of spaces to vent said lens; and
an attachment device attached to the lens so that pressure is maintained upon the seals.

20. The rollable goggle of claim 19 further comprising:
at least one opening in said lens adjacent said seal; and
a clip for securing through said at least one opening in said lens for securing said seal to said lens.

21. The rollable goggle of claim 19 further comprising:
a storage container for the rolled goggles.
